(19) United States
(12) Patent Application Publication
(10) Pub. No.: US 2008/0118962 A1
(43) Pub. Date: May 22, 2008

(54) PROCESS FOR THE SYNTHESIS OF CIS-1,3-DIOLS

(76) Inventors: Michael Paul Burns, Mystic, CT (US); John Wing Wong, East Lyme, CT (US)

Correspondence Address: PFIZER INC., PATENT DEPARTMENT, MS8260-1611, EASTERN POINT ROAD, GROTON, CT, 06340, US

(21) Appl. No.: 11/941,485

(22) Filed: Nov. 16, 2007

Related U.S. Application Data
(60) Provisional application No. 60/866,336, filed on Nov. 17, 2006.

(51) Int. Cl. C12P 7/62

(52) U.S. Cl. 435/135

(57) ABSTRACT

A process for selectively reducing beta-hydroxy ketones, using a ketone reductase obtained from: Monosporium, Rhodococcus, Lechevalieria, Fusarium, Sporidiobolus, Streptomyces, Absidia, or Rhodotorula, to obtain the corresponding cis-1,3-diol. A purified ketone reductase obtained from an organism of the genera Monosporium, Rhodococcus, Lechevalieria, Fusarium, Sporidiobolus, Streptomyces, Absidia, or Rhodotorula.

### FIELD OF THE INVENTION

The present invention relates to a process for preparing cis-1,3-diols. More particularly, to (3R,5R)-tert-butyl 6-cyano-3,5,dihydroxyhexanoate.

### BACKGROUND OF THE INVENTION

Processes for selectively reducing a beta-hydroxy ketone to obtain the corresponding cis-1,3-diol are described in the literature for example: U.S. Pat. No. 6,962,994 also U.S. Pat. No. 6,001,615 describe reducing a beta-hydroxy ketone to obtain the corresponding cis-1,3-diol using ketone reductase expressing organisms.

These cis-diols are valued as intermediates for the preparation of, for example, HMG-CoA reductase inhibitors containing a cis-1,3-diol moiety. These inhibitors are useful as hypolipidemic and hypocholesterolemic agents. This is a widely used method of preparation of such agents for example U.S. Pat. Nos. 4,645,854, 5,354,772, 5,155,251, and 4,970,313. Chemical reduction methods often require hazardous reagents, cryogenic conditions, and complicated workup procedures, and may lack selectivity with respect to producing the desired cis diastereomers. A process using ketone reductase obtained from specific microorganisms to reduce a beta-hydroxy ketone to obtain the corresponding cis-1,3-diol is described in U.S. Pat. No. 6,001,615. However, it is desirable to identify other microorganism that are able to carry out this reaction We have found that reduction of hydroxy-ketones to cis-diols can be carried out with high selectivity and without the use of hazardous reagents using ketone reductases from one or more microorganism of the genera: Monosporium, Rhodococcus, Lechevalieria, Fusarium, Sporidiobolus, Streptomyces, Absidia, or Rhodotorula.

### SUMMARY OF THE INVENTION

A process for producing a cis-1,3-diol comprising the steps of reducing a corresponding beta-hydroxy ketone using a ketone reductase wherein the ketone reductase is obtained from: Monosporium, Rhodococcus, Lechevalieria, Fusarium, Sporidiobolus, Streptomyces, Absidia, or Rhodotorula and;

recovering the desired cis-1,3-diol.

### DETAILED DESCRIPTION

In this invention the term “alkyl” means a straight or branched hydrocarbon radical having from 1 to 10 carbon atoms and includes, for example, methyl, ethyl, n-propyl, isopropyl, n-butyl, secondary-butyl, isobutyl, tertiary butyl (t-butyl), n-pentyl, n-hexyl, n-heptyl, n-octyl, n-nonyl, n-decyl, and the like.

“Purified ketone reductase” or “purified enzyme” means: a preparation derived by removal of some of the materials or the majority of materials not contributing to the desired activity.

“Obtained from” means that the ketone reductase used is provided from an organism in the form of whole cells, modified whole cells, including but not limited to, dead cells, cell lysates, supernatant from cell lysates, or purified enzyme.

The article “a” or “an” as used herein refers to both the singular and plural form of the object to which it refers.

“Halo” means halogens such as fluorine, chlorine, and bromine or iodine atoms.

The compound of formula II is either known in the art or capable of being prepared by methods known in the art, for example in U.S. Pat. No. 5,155,251.

One embodiment of the invention provides a process for producing a cis-1,3-diol comprising the steps of reducing the corresponding beta-hydroxy ketone using a ketone reductase wherein the ketone reductase is obtained from: Monosporium, Rhodococcus, Lechevalieria, Fusarium, Sporidiobolus, Streptomyces, Absidia, or Rhodotorula and; recovering the desired cis-1,3-diol.

In one embodiment the invention provides a process for producing a compound of formula (I)

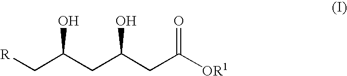

wherein R is halo or —CN; and

### R¹ is alkyl of 1, 2, 3, 4, 5, or 6 carbon atoms;

comprising:
reducing a compound of formula II

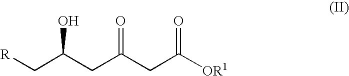

wherein R and R¹ are as defined above, with a ketone reductase obtained from Monosporium, Rhodococcus, Lechevalieria, Fusarium, Sporidiobolus, Streptomyces, Absidia or Rhodotorula and recovering the compound of Formula (I).

In another embodiment of the invention is purified ketone reductase that is obtained from: Monosporium, Rhodococcus, Lechevalieria, Fusarium, Sporidiobolus, Streptomyces, Absidia or Rhodotorula.

In another embodiment of the invention the reductase is provided in the form of whole cells of: Monosporium, Rhodococcus, Lechevalieria, Fusarium, Sporidiobolus, Streptomyces, Absidia or Rhodotorula.

In another embodiment of the invention the ketone reductase is obtained from Monosporium olivaceum v. major, Rhodotorula pilimanae, Rhodococcus rhodochorous, Lechevalieria aerocolonigeses, Fusarium solani, Sporidiobolus johnsonii, Streptomyces violascens, Absidia cylindrospora, Rhodotorula sp., Rhodotorula minuta, Rhodotorula rubra or Rhodotorula mucilaginosa var. mucilaginosa.

In another embodiment of the invention the reductase is in the form of a purified ketone reductase that is obtained from: Monosporium olivaceum v. major, Rhodotorula pilimanae, Rhodococcus rhodochorous, Lechevalieria aerocolonigeses, Fusarium solani, Sporidiobolus johnsonii, Streptomyces violascens, Absidia cylindrospora, Rhodotorula sp., Rhodotorula minuta, Rhodotorula rubra or Rhodotorula mucilaginosa var. mucilaginosa.

In another embodiment of the invention the reductase is provided in the form of whole cells of: Monosporium olivaceum v. major, Rhodotorula pilimanae, Rhodococcus rhodochorous, Lechevalieria aerocolonigeses, Fusarium solani, Sporidiobolus johnsonii, Streptomyces violascens, Absidia cylindrospora, Rhodotorula sp., Rhodotorula minuta, Rhodotorula rubra or Rhodotorula mucilaginosa var. mucilaginosa

In another embodiment of the invention the ketone reductase is obtained from Rhodotorula sp., Rhodotorula minuta, Rhodotorula pilimanae, Rhodotorula rubra or Rhodotorula mucilaginosa var. mucilaginosa.

In another embodiment of the invention the reductase is in the form of a purified ketone reductase that is obtained from: Rhodotorula sp., Rhodotorula minuta, Rhodotorula pilimanae, Rhodotorula rubra or Rhodotorula mucilaginosa var. mucilaginosa.

In another embodiment of the invention the reductase is provided in the form of whole cells of: Rhodotorula sp., Rhodotorula minuta, Rhodotorula pilimanae, Rhodotorula rubra or Rhodotorula mucilaginosa var. mucilaginosa.

In another embodiment of the invention R¹ is tertiary butyl.

In another embodiment of the invention R is —CN.

In another embodiment of the invention R is Chloro or Bromo.

One of skill in the art would recognize that the enzymatic activity and stereoselectivity can be increased using mutagenesis of the DNA of the organism listed above. Suitable methods of mutagenesis are well known in the art, these methods include site-directed mutagenesis or random mutagenesis using Error-prone-PCR. For other methods and a description of their use see, Organic Process Research & Development 2006, 10, 562-571

Cells used in the process of the invention are grown in a suitable nutrient medium. Growth and maintenance conditions for culture of the organisms used in the invention are well known to one of skill in the art.

The reduction may be carried out using whole cells or with ketone reductase that has been purified from whole cells. The conversion of the beta-hydroxy ketone to the corresponding cis-1,3-diol with an isolated ketone reductase must be carried out in the presence of a co-factor, such as nicotinamide adenine dinucleotide (NADH) or nicotinamide adenine dinucleotide phosphate (NADPH) and components for regenerating the co-factor for example: glucose and glucose dehydrogenase. Alternately, the conversion of the beta-hydroxy ketone to the corresponding cis-1,3-diol may be carried out using whole cells of the organism in a nutrient medium, in which case the cells may provide the co-factor regeneration components. The nutrient medium may be that used normally to culture the organism, for example a medium that contains a suitable carbon source. If cell growth during the reaction is desired the medium should contain nitrogen, and phosphorus sources and trace elements. A suitable carbon source is, for example, maltose, sucrose, glucose, polyol (e.g. glycerol, sorbitol), citric acid, or a lower alcohol such as methanol or ethanol. One of skill in the art would be readily able to select an appropriate growth medium for the maintenance and use of the cells.

In one embodiment of the invention, a compound of formula (II) is added to a suspension of live cells in a medium that supports growth of the organism. In another embodiment the compound of formula (II) is added to a suspension of the live cells that lacks one or more nutrients necessary for growth. Dead cells may also be used provided that the necessary enzymes and co-factors are present. The cells may be immobilized on a support.

The process of the invention may be carried out at a pH between of about 3.5 and about 9, preferably between about 6 and about 9, and more preferably between about 6 and about 8, most preferably about 7. Suitable temperatures for the process of the invention are about 10 to about 50° C., preferably about 20 to about 40° C., and more preferably about 25 to about 35° C.

When live cells are used, the process is carried out aerobically. One skilled in the art would be able to select suitable aeration conditions.

Purified enzymes may be isolated using methods well known in the art (e.g. Robert K. Scopes, (1994), Protein Purification: Principles and Practice, Third Edition, Springer-Verlag, New York). These methods may include centrifugation of whole or lysed cells, isolating the enzyme from the supernatant, for example by ion exchange chromatography or by selective precipitation or both.

The following non-limiting example illustrates the inventors preferred method for obtaining the compound of the invention.

### EXAMPLE 1

### Preparation of (3R,5R)-tert-butyl 6-cyano-3,5,dihydroxyhexanoate

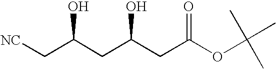

Individual cultures of the organisms: Monosporium olivaceum v. major, Rhodotorula pilimanae, Rhodococcus rhodochorous, Lechevalieria aerocolonigeses, Fusarium solani, Sporidiobolus johnsonii, Debaryomyces marama, Streptomyces violascens, Absidia cylindrospora, Rhodotorula sp., Rhodotorula minuta, Rhodotorula rubra or Rhodotorula mucilaginosa var. mucilaginosa were maintained as frozen stocks at −80° C. For each of the frozen stocks, the stock was thawed and used to inoculate a 300 mL Erlenmeyer flask containing 25 mL of a medium of composition (per liter) glucose (20.0 g), NaCl (5.0 g), yeast extract (5.0 g), soy flour (5.0 g), K₂HPO₄ (5.0 g), pH 7.0.

Cultures were incubated at 29° C. on an orbital shaker at 210 rpm for 48 hours. The entire contents of the Erlenmeyer flask seed culture was used to inoculate a 3 L Fernbach flask that contained 500 mL of the same medium. The Fernbach flask was incubated for 24 hours (for yeast and bacteria) or 48 hours (for fungi and actinomycetes) at 29° C. on an orbital shaker at 210 rpm.

### (R)-tert-butyl 6-cyano-5-hydroxy-3-oxohexanoate

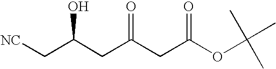

was added to the Fernbach flask (2.0 mL of a 500 g/L stock in dimethyl sulfoxide) to yield an initial concentration of 2.0 g/L. The cultures were incubated for an additional 3 days (bacteria and yeast) or 4 days (fungi and actinomycetes) at 29° C. on an orbital shaker at 210 rpm.

The contents of each culture flask were then extracted two times with 1 L of ethyl acetate. The pooled organic extracts were dried over anhydrous MgSO₄, filtered through a sintered glass funnel, and concentrated under reduced pressure to yield (3R,5R)-tert-butyl 6-cyano-3,5,dihydroxyhexanoate. The extent of conversion of (R)-tert-6-cyano-5-hydroxy-3-oxohexanoate (ketoester) to (3R,5R)-tert-butyl 6-cyano-3,5,dihydroxyhexanoate (cis-diol) and the diastereomeric excess over (3R,5S)-tert-butyl 6-cyano-3,5-dihydroxyhexanoate (trans-diol) was determined using HPLC (high performance liquid chromatography). The conditions for HPLC are described below:

### HPLC: Waters 2790 Separations Module

### Column: Inertsil C₈, 5 micron particle size, 4.6 mm×250 mm, GL Sciences, Inc.

Solvent: water:acetonitrile (80:20, v/v)

### Flow Rate: 1.0 mL/minute

### Temperature: 30° C.

### Detection: Refractive Index (Waters Model 2414 Refractive Index Detector)

The retention times of the trans diol, cis diol, and ketoester were 16.9 minutes, 17.8 minutes, and 27.1 minutes respectively. The results obtained are summarized in Table 1.

TABLE 1

| | | | | % |
|---|---|---|---|---|
| | | Organism | Conversion | diastereomeric |
| Strain No. | Organism Name | Class | (%) | excess* |
| ATCC | Monosporium olivaceum v. major | fungi | 4.0 | 90.3 |
| 36300A | | | | |
| UC7014 | Rhodotorula pilimanae | yeast | 11.9 | 97.9 |
| ATCC | Rhodococcus rhodochrous | bacteria | 2.6 | 67.1 |
| 21766 | | | | |
| ATCC | Lechevalieria aerocolonigenes | bacteria | 2.6 | 71.6 |
| 39243 | | | | |
| ATCC | Fusarium solani | fungi | 4.5 | 94.1 |
| 12823 | | | | |
| ATCC | Sporidiobolus johnsonii | yeast | 12.8 | 95.2 |
| 16039 | | | | |
| ATCC | Debaryomyces marama | yeast | 8.6 | 98.7 |
| 11627 | | | | |
| ATCC | Streptomyces violascens | actinomycete | 8.4 | 66.1 |
| 31560 | | | | |
| ATCC | Absidia cylindrospora | fungi | 2.6 | 88.6 |
| 11516 | | | | |
| UC1271 | Rhodotorula sp. | yeast | 14.2 | 96.9 |
| ATCC | Rhodotorula minuta | yeast | 11.1 | 98.0 |
| 02776 | | | | |
| UC5131 | Rhodotorula rubra | yeast | 11.4 | 97.3 |
| ATCC | Rhodotorula minuta | yeast | 19.7 | 98.8 |
| 36236 | | | | |
| ATCC | Rhodotorula mucilaginosa var. | yeast | 14.5 | 98.5 |
| 32762 | mucilaginosa | | | |

((amount cis diol − amount trans diol)/(amount cis diol + amount trans diol)) × 100%

What is claimed:

1. A process of making a cis-1,3-diol comprising the steps of reducing a corresponding beta-hydroxy ketone using a ketone reductase wherein the ketone reductase is obtained from: Monosporium, Rhodococcus, Lechevalieria, Fusarium, Sporidiobolus, Streptomyces, Absidia, or Rhodotorula and;
recovering the desired cis-1,3-diol.

2. A process for making a compound of Formula (I); 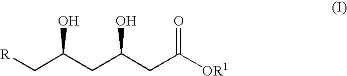
wherein R is halo or —CN; and
R¹ is alkyl of 1, 2, 3, 4, 5, or 6 carbon atoms;
comprising:
reducing a compound of formula II 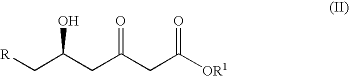
wherein R and R¹ are as defined above, with a ketone reductase obtained from: Monosporium, Rhodococcus, Lechevalieria, Fusarium, Sporidiobolus, Streptomyces, Absidia, or Rhodotorula and;
recovering the compound of Formula (I).

3. The process according to claim 2, wherein the ketone reductase is provided in the form of whole cells.

4. The process according to claim 2 wherein the ketone reductase is provided in the form of a purified ketone reductase.

5. The process according to claim 2 wherein the ketone reductase is obtained from Monosporium olivaceum v. major, Rhodotorula pilimanae, Rhodococcus rhodochorous, Lechevalieria aerocolonigeses, Fusarium solani, Sporidiobolus johnsonii, Streptomyces violascens, Absidia cylindrospora, Rhodotorula sp., Rhodotorula minuta, Rhodotorula rubra or Rhodotorula mucilaginosa var. mucilaginosa.

6. The process according to claim 5, wherein the ketone reductase is provided in the form of whole cells.

7. The process according to claim 2 wherein R¹ is tertiary butyl.

8. The process according to claim 5 wherein R is —CN and R¹ is tertiary butyl.

9. The process according to claim 5 wherein the ketone reductase is provided in the form of a purified enzyme.

10. A purified ketone reductase obtained from Monosporium, Rhodococcus, Lechevalieria, Fusarium, Sporidiobolus, Streptomyces, Absidia, or Rhodotorula.

11. The purified ketone reductase of claim 10 wherein the ketone reductase is obtained from Monosporium olivaceum v. major, Rhodotorula pilimanae, Rhodococcus rhodochorous, Lechevalieria aerocolonigeses, Fusarium solani, Sporidioboluss johnsonii, Streptomyces violascens, Absidia cylindrospora, Rhodotorula sp., Rhodotorula minuta, Rhodotorula rubra and Rhodotorula mucilaginosa var. mucilaginosa.
